(19) United States
(12) Patent Application Publication
(10) Pub. No.: US 2018/0318120 A1
(43) Pub. Date: Nov. 8, 2018

(54) U' KNEE-QUE ICE PACK

(71) Applicant: Charles Dexter Oliver, Orlando, FL (US)

(72) Inventor: Charles Dexter Oliver, Orlando, FL (US)

(21) Appl. No.: 15/275,051

(22) Filed: May 3, 2017

(51) Int. Cl. A61F 5/01; A61F 7/10

(52) U.S. Cl. CPC A61F 5/0109; A61F 2007/0042; A61F 7/10

(57) ABSTRACT

A Knee Brace with an Ice Pouch or Retainer for liquids or solid coolants that has 3 adjustable Velcro straps which compresses, secures, and conforms to injured knee. The Device has 3 attached hooks for the straps to pass through. The Ice Pouch of the Knee brace has a waterproof zipper. While in use, the Pouch is filled with ice or a coolant insert. When zipped completely, the pouch will contain and retain the ice or coolant. Pulling the Velcro straps through the hooks. The hooks are to help the device conform to the injured knee. The Knee brace, made of neoprene material conforms around the back of the injured knee about 8 inches above the back of the knee and 8 inches below the back of the knee then pulling the adjustable Velcro straps with comfortable tension to fit through the hooks to allow mobility while minimizing swelling with a quick method of icing the injured knee.

### DETAILED DESCRIPTION OF THE U′ KNEE-QUE ICEOPEDIC SUPPORT

FIG. 1 Front Side

The top width is about 21 inches. The left side height is about 18 inches. The right side height is about 18 inches. The top strap is about 28 inches and will pull across the front to be held in place by the Velcro. The center strap is about 25 inches and will pull across the front to be held in place by the Velcro. The bottom strap is about 28 inches and will pull across the front to be held in place by the Velcro. The straps will allow the U′Knee-Que Iceopedic Support to conform and fit most sizes. The center front width is about 20 inches. The bottom front width is about 16 inches. The front of the plastic ice pouch is about 8 inches in height on the left front and about 8 inches in height on the right front. The bottom front of the ice pouch is about 7 inches in width. The #6 molded zipper is at the top and front of the plastic ice pouch and is about 9 inches from left to right made of Nylon Laminated Neoprene.

FIG. 2 Back Side

The backside top width is about 21 inches. The top strap backside view (is folded across the front and is unseen) is about 28″ made of stretch loop tape (also called Vel stretch by the Velcro company). The center strap backside view (is folded across the front and is unseen) is about 25″ made of stretch loop tape (also called Vel stretch by the Velcro Company). The bottom strap backside view (is folded across the front and is unseen) is about 28″ made wide stretch loop tape (also called Vel stretch by the Velcro Company). The backside center width is about 20″. The backside bottom width is about 16″. The backside of the plastic ice pouch is about 8″ in height on the left side and about 8″ in height on the right side. The bottom backside of the ice pouch is about 7″ in width. The top, bottom, left side, and right side of the ice pouch is attached to the main fabric which is about 2 mm. one side Nylon laminated Neoprene and one side fair skin Transparent PVC, 16 gage, dark grey color.

FIG. 3

References are now made to FIG. 3 drawing, wherein like numerals are used to designate like parts throughout. The U′Knee-Que Iceopedic Ice Pack/Knee Support is designated as 1 in “FIG. 3”, is illustrated as the “Main Body” and is about 22″×18″ made of Rubber Neoprene laminated on the outer side only. The functionality is to be able to apply ice immediately to an injured knee without being immobile. The numeral 2 being illustrated as a sewn in coolant bag that is about 10×10 inches made of Polyvinyl Chloride (PVC). As illustrated in 3 The top width of the device is about 22″ goes up mid-thigh to support the weight of the ice. Illustrated in numeral 4 the total height of the embodiment is about 18″ to provide coverage for the main areas. Illustrated in 5 of numeral 1, the bottom width at mid-calf is about 14″ made of Rubber Neoprene laminated on the outer side to support the weight of the ice. The numeral 6, there is a top corner radius that is about 1¼″ to facilitate continued support of the weight of the ice. The numeral 7, there is inside binding tape edge for aesthetics which is 80% Nylon and 20% Spandex. The numeral 8, there is about a 4½″ dart length for contour. The numeral 9, the dart stitching width is about ¼″. The dart stitching is 100% polyester which allows for flexibility and stretching. The numeral 10, the water proof zipper allows for easy access to fill the plastic coolant/ice pouch (bag). The numeral 11, the nylon zipper tape holds the zipper teeth and allows sewing. The numeral 12, the zipper bottom stitching is about ⅛″ which attaches the zipper to the ice bag. The numeral 13, the binding tape outer edge is made of 80% nylon and 20% spandex, is for aesthetics. The numeral 14, the nylon binding tape top stitching secures the binding tape edges. The numeral 15, the binding tape is 80% Nylon and 20% Spandex gives the device a clean aesthetic finish. The numeral 16, there is an inverted pleat made with Polyvinyl Chloride (PVC) with about a 2″ finished width to provide more fullness for ice if needed. The numeral 17, there is an inverted pleat outer fold made of Polyvinyl Chloride (PVC) to provide extra fullness for ice. The numeral 18, there is a half pleat finished width of about 1″ made of Polyvinyl Chloride (PVC) to provide extra fullness for ice. The numeral 19, there is an inverted pleat inner fold to provide fullness for extra ice made of polyvinyl chloride (PVC). The numeral 20, there is about a ¼″ plastic loop frame made of polyacetal to support the loop tape. The numeral 21 there is a plastic loop opening that allows you to thread the tape through for sewing. The numeral 22, the mid-thigh loop tape is Nylon and is about 26″×1½″ and wraps around the mid-thigh to secure the top. The numeral 23, the mid-thigh hook tape is nylon and is about ⅛″ stitching and is attached to loop tape for closure. The numeral 24, the mid-thigh nylon hook tape is about 3½″×1½″ and hooks onto the loop tape for closure. The numeral 25, the loop tape stitching at the plastic loop is about ⅛″ and attaches the loop tape to the main body (numeral 1). The numeral 26, the nylon knee part hook tape is about 2¾×½″ and attaches to loop tape for closure. The numeral 27 the knee part hook tape stitch is about ⅛″ and is nylon and attaches to loop tape for closure. The numeral 28, the knee part loop tape is nylon and is about 10″ and attaches to loop tape for closure. The numeral 29, the mid-calf part loop tape is about ⅛″ stitching and attaches to loop tape for closure. The numeral 30, the mid-calf part nylon hook tape is about 21″ and secures the device at mid-calf. The numeral 31, the mid-calf part nylon hook tape is about 3½″×1½ ″ and attaches to loop tape for closure. The numeral 32 decreases by about 2¼″ for contour. The numeral 33, the fused edge on the ice bag is about ⅜″ made of polyvinyl chloride (PVC) which prevents melted ice from leaking.

### BRIEF DESCRIPTION OF DRAWINGS

FIG. 1 illustrates a front view of the U′ Knee-Que Iceopedic Pack/Knee Support

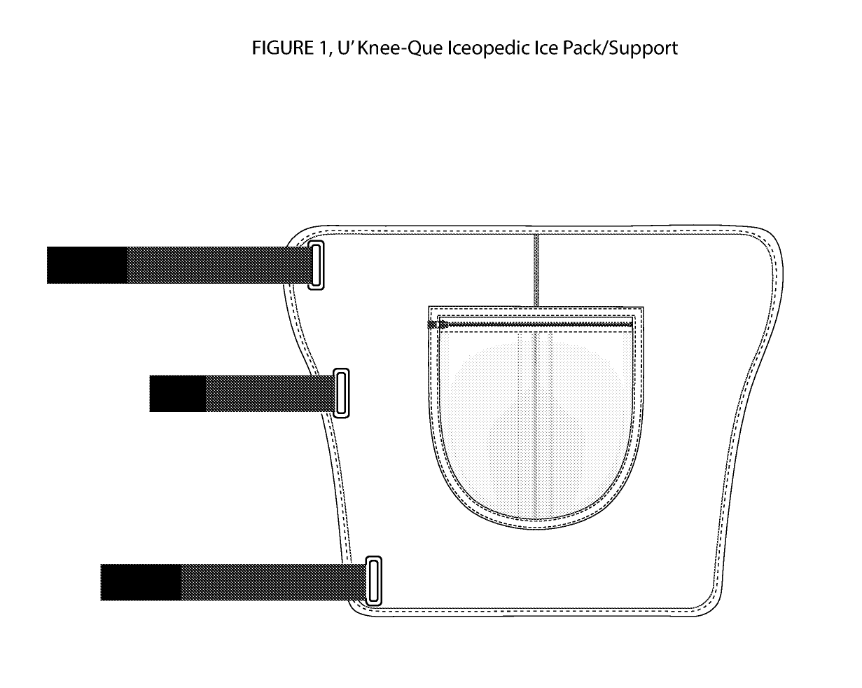

FIG. 2 illustrates a back view of the U′ Knee-Que Iceopedic Ice Pack/Knee Support

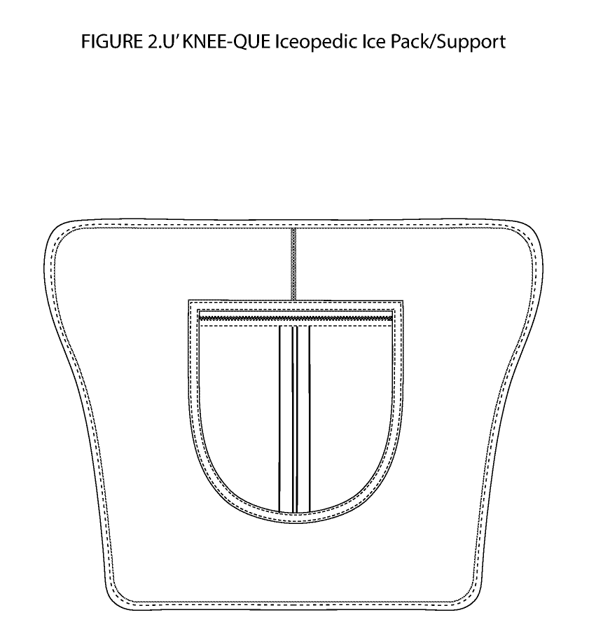

FIG. 3 illustrates a full view of the U′ Knee-Que Iceopedic Ice Pack/Knee Support

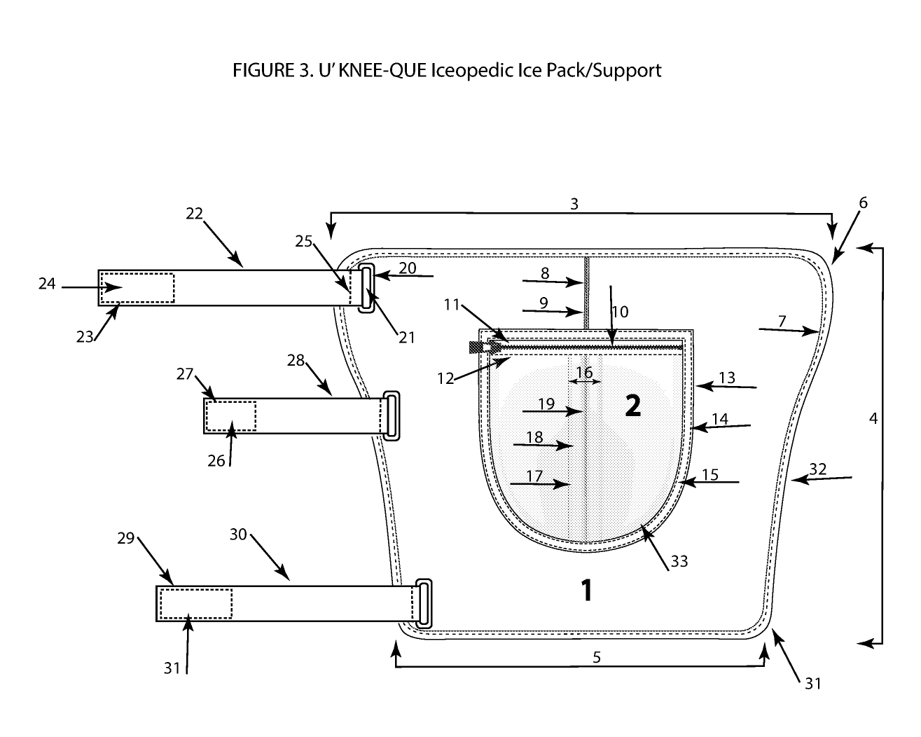

1. A Knee Brace/Ice Pouch comprising of 3 adjustable velcro straps along with 3 hooks, A waterproof zipper secured to said frontal portion of said Ice Pouch of the Knee Brace/Ice Pouch.
